(19) United States
(12) Patent Application Publication
(10) Pub. No.: US 2017/0209354 A1
(43) Pub. Date: Jul. 27, 2017

(54) MULTILAMELLAR VESICLE PREPARATION CONTAINING ACYL BASIC AMINO ACID DERIVATIVE AND PHYSIOLOGICALLY ACTIVE SUBSTANCE

(71) Applicant: AJINOMOTO CO., INC., Tokyo (JP)

(72) Inventor: Shun KOBAYASHI, Kawasaki-shi (JP)

(73) Assignee: AJINOMOTO CO., INC., Tokyo (JP)

(21) Appl. No.: 15/409,219

(22) Filed: Jan. 18, 2017

(30) Foreign Application Priority Data
Jan. 21, 2016 (JP) 2016-009785

(51) Int. Cl. A61K 8/42; A61Q 19/02; A61K 8/67; A61K 8/97; A61K 8/49; A61K 8/34; A61K 8/31; A61K 8/02; A61K 8/37

(52) U.S. Cl. CPC A61K 8/42; A61K 8/0245; A61Q 19/02; A61K 8/678; A61K 8/37; A61K 8/676; A61K 8/494; A61K 8/34; A61K 8/31; A61K 8/97; A61K 8/49; A61K 2800/596; A61K 2800/594

(57) ABSTRACT

The present invention aims to provide a multilamellar vesicle preparation which can be produced conveniently and is superior in stability. A multilamellar vesicle preparation containing component (A): a compound represented by the formula (1)

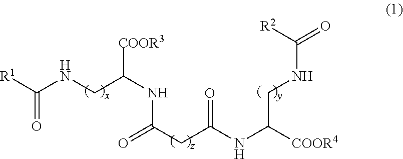

wherein each symbol is as described in the SPECIFICATION, or a salt thereof, component (B): a physiologically active substance, and component (C): water.

### BACKGROUND OF THE INVENTION

Field of the Invention

The present invention relates to a multilamellar vesicle preparation containing an acyl basic amino acid derivative, a physiologically active substance and water, and an external preparation and a cosmetic containing the preparation.

Discussion of the Background

In recent years, under the concept of drug delivery system (DDS), a technique for transporting to or absorbing/holding a drug at an affected part to enhance or sustain drug efficacy is attracting attention. From the technical aspect, an administration method by transdermal absorption is attracting much attention, and a liposome preparation which is known as a useful carrier for transporting a substance to the internal tissues is of particular interest (non-patent document 1).

Liposome is a complex similar to a lipid bilayer membrane of a cell membrane and is composed of phospholipid having a hydrophilic part and a hydrophobic part in a molecule. It has been recently known that multilamellar vesicles having a structure in which bilayer membranes are wound multiple times in the inner structure can also be applied to DDS.

Phospholipids which are constituent components of cellular membrane, ceramide which is the main component of the skin stratum corneum, and the like are generally used as the base for producing liposome preparations. However, these phospholipids, ceramides and the like are substances poorly soluble in water, and advanced techniques are required for forming liposome preparations.

For example, organic solvents such as chloroform and the like are used for obtaining a liposome preparation (patent document 1). However, such use causes problematic influence on the human body and makes operations complicated. In addition, a liposome preparation using phospholipid and ceramide (patent document 2) has problems in that it develops an odor and a color problem derived from lecithin (phospholipid), requires a high-pressure treatment to achieve stability over time, requires use of a limited apparatus and the like.

Incidentally, a compound represented by the following formula:

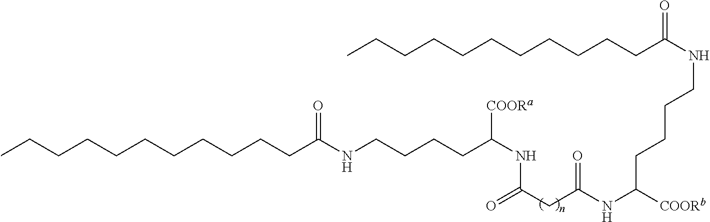

wherein R^{a} and R^{b} are each a hydrogen atom or an alkyl group, and n is an integer of 0 to 12,
or a salt thereof (hereinafter to be also referred to as “lauroyl amino acid derivative”) has been reported to be useful for gelling or solidifying water and liquid organic medium (patent document 3, non-patent document 2 and non-patent document 3 etc.).

### DOCUMENT LIST

### Patent Documents

[patent document 1] JP-A-2000-63265
[patent document 2] JP-A-2014-208626
[patent document 3] JP-A-2004-323505

### Non-Patent Documents

[non-patent document 1] YAKUGAKU ZASSHI 128(2) 185-186 (2008)
[non-patent document 2] Org. Biomol. Chem., 2003, 1, 4124-4131
[non-patent document 3] New J. Chem., 2005, 29, 1439-1444

### SUMMARY OF THE INVENTION

### Problems to be Solved by the Invention

The present invention aims to provide a preparation that can be produced with ease without using an organic solvent such as chloroform and the like or phospholipid, and maintains a stable multilamellar vesicle structure over time.

### Means of Solving the Problems

The present inventors have conducted intensive studies in an attempt to achieve the aforementioned object and found that a multilamellar vesicle is unexpectedly formed from component (A) a compound represented by the following formula (1) (hereinafter to be also referred to as “compound (1)”) or a salt thereof, (B) at least one kind of physiologically active substance and (C) water alone, and the multilamellar vesicle is stable over time and can be produced easily, which resulted in the completion of the present invention.

Accordingly, the present invention provides the following.

[1] A multilamellar vesicle preparation comprising component
(A): a compound represented by the formula (1)

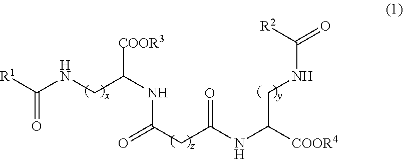

wherein

R¹ and R² are each independently an alkyl group having 5-21 carbon atoms or an alkenyl group having 5-21 carbon atoms,

R³ and R⁴ are each independently a hydrogen atom, an alkyl group having 1-22 carbon atoms or an alkenyl group having 2-22 carbon atoms,

z is an integer of not less than 0, and

x and y are each independently an integer of 2-4 or a salt thereof;

component (B): a physiologically active substance, and
component (C): water.
[2] The preparation of [1], wherein the component (A) is a compound of the aforementioned formula (1) wherein z is an integer of 0-10, or a salt thereof.
[3] The preparation of [1] or [2], wherein the component (A) is a compound of the aforementioned formula (1) wherein z is 0.7 or 8, or a salt thereof.
[4] The preparation of any of [1]-[3], wherein the component (A) is a compound of the aforementioned formula (1) wherein x and y are each 4, or a salt thereof.
[5] The preparation of any of [1]-[4], wherein the component (A) is a compound of the aforementioned formula (1) wherein R¹ and R² are each independently a straight chain alkyl group having 5-15 carbon atoms, or a salt thereof.
[6] The preparation of any of [1]-[5], wherein the component (A) is a compound of the aforementioned formula (1) wherein both R³ and R⁴ are hydrogen atoms, or a salt thereof.
[7] The preparation of any of [1]-[6], wherein the component
(A) is bis(N^{ε}-lauroyl-L-lysine)sebacoyl amide or a salt thereof.
[8] The preparation of any of [1]-[7], wherein the physiologically active substance as component (B) is at least one kind selected from the group consisting of a whitening agent, an antioxidant, an anti-inflammatory agent, an algefacient, and an animal or plant-derived component.
[9] The preparation of any of [1]-[8], wherein the physiologically active substance as component (B) is at least one kind selected from the group consisting of tocopheryl acetate, acetyl ethylcarboxyl methylthiazolidine carboxylic acid, retinyl palmitate, ascorbyl tetra-2-hexyldecanoate, allantoin, menthol, guaiazulene and an oil-soluble licorice extract.
[10] The preparation of any of [1]-[9], comprising 0.0001-2 parts by weight of component (B) per 1 part by weight of component (A).
[11] The preparation of any of [1]-[10], comprising 30-200 parts by weight of component (C) per 1 part by weight of component (A).
[12] An external preparation comprising the preparation of any of [1]-[11].
[13] A cosmetic comprising the preparation of any of [1]-[11].

### Effect of the Invention

According to the present invention, a multilamellar vesicle preparation can be produced conveniently by using compound (1), without using an organic solvent such as chloroform and the like.

According to the present invention, since multilamellar vesicle can be provided without adding phospholipid, a multilamellar vesicle preparation superior in preservation stability and free of an odor and a color problem derived from phospholipid can be provided.

According to the present invention, moreover, production is conveniently performed without a high-pressure treatment and the like which are necessary for adding phospholipid.

According to the present invention, moreover, since various physiologically active substances can be supported in a multilamellar vesicle, it can be used as a high-performance material for pharmaceutical product, food and drink, cosmetic, quasi-drug, feed and the like.

### BRIEF DESCRIPTION OF THE DRAWINGS

FIG. 1 is a polarized microscope photograph of a multilamellar vesicle. The inside of a circle is a maltese cross image indicating a multilamellar vesicle (see schematic showing). In the Figure, the scale is 100 μm

FIG. 2 is a polarized microscope photograph of Example 2. In the Figure, the scale is 100 μm

FIG. 3 is a polarized microscope photograph of Comparative Example 2. In the Figure, the scale is 100 μm

### DETAILED DESCRIPTION OF THE PREFERRED EMBODIMENTS

The multilamellar vesicle preparation of the present invention characteristically contains component (A): a compound represented by the formula (1)

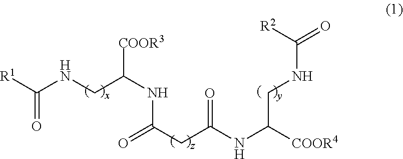

wherein

R¹ and R² are each independently an alkyl group having 5-21 carbon atoms or an alkenyl group having 5-21 carbon atoms,

R³ and R⁴ are each independently a hydrogen atom, an alkyl group having 1-22 carbon atoms or an alkenyl group having 2-22 carbon atoms,

Z is an integer of not less than 0, and

x and y are each independently an integer of 2-4 or a salt thereof;

component (B): at least one kind of a physiologically active substance, and
component (C): water.

The “multilamellar vesicle” means a spherical structure having a structure in which bilayer membranes are wound multiple times in the inner structure, and the “multilamellar vesicle preparation” means a preparation having such structure.

The embodiment of the present invention is described in detail in the following.

### 1. Component (A): A Compound Represented by the Formula (1) (Compound (1)) or a Salt Thereof

R¹ and R² are each independently an alkyl group having 5-21 carbon atoms or an alkenyl group having 5-21 carbon atoms.

An alkyl group having 5-21 carbon atoms means a straight chain or branched alkyl group having 5-21 carbon atoms. Specific examples thereof include pentyl group, isopentyl group, neopentyl group, hexyl group, isohexyl group, neohexyl group, heptyl group, isoheptyl group, neoheptyl group, octyl group, isooctyl group, nonyl group, isononyl group, decyl group, isodecyl group, undecyl group, dodecyl group, tridecyl group, tetradecyl group, pentadecyl group, hexadecyl group, heptadecyl group, octadecyl group, nonadecyl group, icosyl group and the like.

An alkenyl group having 5-21 carbon atoms means a straight chain or branched alkenyl group having 5-21 carbon atoms. Specific examples thereof include pentenyl group, hexenyl group, heptenyl group, octenyl group, nonenyl group, decenyl group, undecenyl group, dodecenyl group, tridecenyl group, tetradecenyl group, pentadecenyl group, hexadecenyl group, heptadecenyl group, octadecenyl group, nonadecenyl group, icosenyl group and the like.

An alkyl group having 5-15 carbon atoms means a straight chain or branched alkyl group having 5-15 carbon atoms. Specific examples thereof include pentyl group, hexyl group, heptyl group, octyl group, nonyl group, decyl group, undecyl group, dodecyl group, tridecyl group, tetradecyl group, pentadecyl group and the like.

An alkyl group having 7-11 carbon atoms means a straight chain or branched alkyl group having 7-11 carbon atoms. Specific examples thereof include heptyl group, octyl group, nonyl group, decyl group, undecyl group and the like.

Preferably, R¹ and R² are each independently an alkyl group having 5-15 carbon atoms, more preferably each independently an alkyl group having 7-11 carbon atoms.

Preferably, R¹ and R² are each a straight chain alkyl group. Furthermore, R¹ and R² are preferably the same.

R³ and R⁴ are each independently a hydrogen atom, an alkyl group having 1-22 carbon atoms or an alkenyl group having 2-22 carbon atoms.

An alkyl group having 1-22 carbon atoms means a straight chain or branched alkyl group having 1-22 carbon atoms. Specific examples thereof include methyl group, ethyl group, propyl group, isopropyl group, butyl group, isobutyl group, sec-butyl group, tert-butyl group, pentyl group, isopentyl group, neopentyl group, hexyl group, isohexyl group, neohexyl group, heptyl group, isoheptyl group, neoheptyl group, octyl group, isooctyl group, nonyl group, isononyl group, decyl group, isodecyl group, undecyl group, dodecyl group, tridecyl group, tetradecyl group, pentadecyl group, hexadecyl group, heptadecyl group, octadecyl group, nonadecyl group, icosyl group and the like.

An alkenyl group having 2-22 carbon atoms means a straight chain or branched alkenyl group having 2-22 carbon atoms. Specific examples thereof include ethenyl group, 1-propenyl group, 2-propenyl group, 1-butenyl group, 2-butenyl group, 3-butenyl group, pentenyl group, hexenyl group, heptenyl group, octenyl group, nonenyl group, decenyl group, undecenyl group, dodecenyl group, tridecenyl group, tetradecenyl group, pentadecenyl group, hexadecenyl group, heptadecenyl group, octadecenyl group, nonadecenyl group, icosenyl group and the like.

Preferably, both R³ and R⁴ are hydrogen atoms.

z is an integer of not less than 0.

z is preferably an integer of 0-10, more preferably 7 or 8.

x and y are each independently an integer of 2-4.

Preferably, both x and y are 4.

As a compound represented by the formula (1), preferably, the following compounds can be mentioned.

### (Compound A)

A compound wherein R¹ and R² are each independently a straight chain alkyl group having 5-15 carbon atoms,

both R³ and R⁴ are hydrogen atoms,

z is an integer of 0-10, and

both x and y are 4.

### (Compound B)

A compound wherein both R¹ and R² are straight chain alkyl groups having 5-15 carbon atoms,

both R³ and R⁴ are hydrogen atoms,

z is 7 or 8, and

both x and y are 4.

### (Compound C)

A compound wherein both R¹ and R² are straight chain alkyl groups having 7-11 carbon atoms,

both R³ and R⁴ are hydrogen atoms,

z is 7 or 8, and

both x and y are 4.

Specific examples of a compound represented by the formula (1) include
bis(N^{ε}-lauroyl-L-lysine)sebacoyl amide, and
bis(N^{ε}-octanoyl-L-lysine)sebacoyl amide,
   or a salt thereof.

A salt of a compound represented by the formula (1) is not particularly limited. Examples thereof include alkali metal salts such as sodium salt, potassium salt and the like, alkaline earth metal salts such as calcium salt, magnesium salt and the like, inorganic salts such as aluminum salt, salt with zinc and the like, and organic salts such as organic amine salts such as ammonium salt, monoethanolamine salt, diethanolamine salt, triethanolamine salt and the like, basic amino acid salts such as arginine salt, lysine salt and the like, and the like. One kind of these may be used, or two or more kinds selected from the above-mentioned group may be used in a mixture. From the aspects of easy availability, handling property and the like, alkali metal salt, organic amine salt, or basic amino acid salt is preferable, and sodium salt and potassium salt are particularly preferable.

Compound (1) is a known compound, and can be produced by a method known per se or a method analogous thereto (JP-A-2004-323505, Org. Biomol. Chem., 2003, 1, 4124-4131, New J. Chem., 2005, 29, 1439-1444 etc.).

The content of component (A) is generally 0.1-10 parts by weight, preferably 0.2-5 parts by weight, more preferably 0.2-3 parts by weight, per 100 parts by weight of a preparation containing multilamellar vesicle of the present invention.

### 2. Component (B): Physiologically Active Substance

The “physiologically active substance” in the present specification is not limited as long as it is a physiologically active substance that can be applied to conventional liposomes and multilamellar vesicles.

The physiologically active substance may be any of water-soluble, oil-soluble, and amphiphilic substances, and an oil-soluble or amphiphilic substance is preferable, and an oil-soluble substance is more preferable.

Examples of the physiologically active substance include whitening agent, antioxidant, anti-inflammatory agent, algefacient, animal or plant-derived component and the like. However, water-soluble moisturizing components are excluded.

Examples of the whitening agent include arbutin, kojic acid, ascorbic acid, ascorbic acid derivatives such as ascorbyl tetra-2-hexyldecanoate and the like, tranexamic acid, hinokitiol, N-acetyl-2-methylthiazoline-2,4-dicarboxylic acid-2-ethyl ester (hereinafter acetyl ethylcarboxyl methylthiazolidine carboxylic acid) and the like.

Examples of the antioxidant include vitamin E, vitamin E derivatives such as tocopheryl acetate and the like, retinol, retinol derivatives such as retinyl palmitate and the like, γ-oryzanol and the like.

Examples of the anti-inflammatory agent include glycyrrhizic acid, glycyrrhizic acid derivative, allantoin, azulene, guaiazulene, aminocaproic acid, hydrocortisone and the like.

Examples of the algefacient include menthol, camphor and the like.

Examples of the animal or plant-derived component include animal or plant itself, plant extract component, generally, edible plant or plant processed product and component derived from an animal. Specific examples thereof include pyracantha fortuneana fruit extract, whey, nicotinic acid amide, diisopropyl amine dichloroacetic acid, mevalonic acid, γ-aminobutyric acid, capsicum annuum fruit extract, ginger tincture, cantharides tincture, althea extract, aloe extract, apricot kernel extract, turmeric extract, Oolong tea extract, sea water-dried product, hydrolyzed wheat powder, hydrolyzed silk, swertia kaponica extract, carrot extract, cucumber extract, gentiana lutea extract, yeast extract, rice germ oil, Russian comfrey extract, soapwort extract, pehmn extract, shikon extract, Japanese white birch extract, western mint extract, swertia kaponica extract, bisabolol, propolis, loofah extract, bodyje extract, hop extract, Aesculus hippocastanum extract, soapberry extract, Melissa officinalis extract, eucalyptus extract, strawberry geranium extract, rosemary extract, roman camomile extract, royal jelly extract, oil-soluble licorice extract, sea weed, rice bran, Aurantii Nobilis Pericarpium, Angelica acutiloba, peach leaf ground product and the like.

Two or more of the above-mentioned physiologically active substances may be used in combination. In the present invention, the physiologically active substance is preferably tocopheryl acetate, acetyl ethylcarboxyl methylthiazolidine carboxylic acid, retinyl palmitate, ascorbyl tetra-2-hexyldecanoate, allantoin, menthol, guaiazulene, oil-soluble licorice extract, arbutin, kojic acid, ascorbic acid, tranexamic acid, vitamin E, retinol, glycyrrhizic acid derivative or nicotinic acid amide, more preferably tocopheryl acetate, acetyl ethylcarboxyl methylthiazolidine carboxylic acid, retinyl palmitate, ascorbyl tetra-2-hexyldecanoate, allantoin, menthol, guaiazulene or oil-soluble licorice extract.

The content of component (B) is generally 0.001-20 parts by weight, preferably 0.002-10 parts by weight, more preferably 0.002-5 parts by weight, per 100 parts by weight of a preparation containing the multilamellar vesicle of the present invention.

A preparation containing the multilamellar vesicle of the present invention generally contains 0.0001-2 parts by weight, preferably 0.0002-1 part by weight, more preferably 0.0002-0.5 parts by weight, of component (B) per 1 part by weight of component (A). The multilamellar vesicle preparation can be stably preserved within these ranges.

### 3. Component (C): Water

Water in the present invention is not particularly limited as long as it can be used for food, cosmetics and the like. For example, purified water, sterilization water, tap water, hard water, soft water, natural water, sea water, deep ocean water, electrolytic alkali ion water, electrolytic acidic ion water, ion water, cluster water and the like can be mentioned.

The water may contain preservative, isotonicity agent and the like as necessary. Examples of the preservative include parabens, chlorobutanol, benzyl alcohol, propylene glycol and the like. As the isotonicity agent, glycerin, glucose, sodium chloride and the like can be mentioned.

The content of water the present invention is generally 30-99 parts by weight, preferably 40-99 parts by weight, more preferably 50-98.5 parts by weight, per 100 parts by weight of the multilamellar vesicle preparation of the present invention.

The multilamellar vesicle preparation of the present invention generally contains 30-200 parts by weight, preferably 40-199 parts by weight, more preferably 50-199 parts by weight, of component (C) per 1 part by weight of component (A). The multilamellar vesicle preparation can be stably preserved within these ranges.

The multilamellar vesicle preparation of the present invention may contain one or more kinds of multilamellar vesicle forming aids (membrane stabilizer), surfactants such as non-ionic surfactant and the like, polyol, polymer, oil agent, powder and the like as necessary.

A multilamellar vesicle forming aid is not particularly limited as long as it is a substance having a function to aid compound (1) to form a multilamellar vesicle structure and increase stability of the obtained multilamellar vesicle structure over time. Specific examples thereof include sterols such as sterol, stigmasterol, lanosterol, ergosterol and the like, fatty acid ester of the sterols (e.g., cholesteryl isostearate), and alkylether of the sterols, esters of saturated or unsaturated, straight chain or branched fatty acid such as lauric acid, myristic acid, palmitic acid, stearic acid, oleic acid (e.g., phytosteryl/octyldodecyl lauroyl glutamate)) and the like.

The content of the multilamellar vesicle forming aid in the present invention is generally 0-10 parts by weight, preferably 0.1-8 parts by weight, per 100 parts by weight of the multilamellar vesicle preparation of the present invention.

Polyol means a straight chain or branched polyvalent alcohol having two or more hydroxyl groups in a molecule, and having two or more carbon atoms (preferably, having 2-6 carbon atoms). Specific examples thereof include 1,3-propanediol, propylene glycol, dipropyleneglycol, ethylene glycol, diethylene glycol, polyethylene glycol, isopreneglycol, 1,3-butanediol(1,3-butyleneglycol), 2,3-butanediol, 1,4-butanediol, 2-butene-1, 4-diol, 1,5-pentanediol, glycerin, diglycerin, triglycerin, polyglycerin, trimethylolpropane, erythritol, pentaerythritol, sorbitol, maltitol, lactose, fructose, maltose, sorbitan, glucose, arabitol, xylitol, mannitol and the like. Of these, 1,3-propanediol, propylene glycol, dipropylene glycol, ethylene glycol, diethylene glycol, polyethylene glycol, isopreneglycol, 1,3-butanediol, 2,3-butanediol and 1,4-butanediol are preferable, and 1,3-propanediol, dipropyleneglycol, 1,3-butanediol, glycerin, sorbitol and the like are more preferable.

One kind of these may be used singly or two or more kinds thereof may be used in combination.

The content of polyol in the present invention is generally 0-30 parts by weight, preferably 1-20 parts by weight, per 100 parts by weight of the multilamellar vesicle preparation of the present invention.

Examples of the non-ionic surfactant include polyglyceryl fatty acid ester, sorbitan fatty acid ester (polyoxyethylene(20)sorbitan oleic acid ester (polysorbate 80) and the like), polyoxyethylene fatty acid ester, polyoxyethylene hydrogenated castor oil, PEG-40 hydrogenated castor oil PCA isostearate and, sucrose fatty acid ester and the like. One kind of these may be used singly or two or more kinds thereof may be used in combination.

The content of the non-ionic surfactant in the present invention is generally 0-10 parts by weight, preferably 0.1-5 parts by weight, per 100 parts by weight of the multilamellar vesicle preparation of the present invention.

The production method of the multilamellar vesicle preparation of the present invention includes the following.

### (When Physiologically Active Substance is Lipophilic)

Component (B) which is a lipophilic physiologically active substance and, where necessary, a non-ionic surfactant and/or a multilamellar vesicle forming aid are heated and mixed to completely and uniformly dissolve them (oil-soluble component). The heating temperature is generally 60-80° C., preferably 70-80° C. Then, component (A) and polyol as necessary are added to water as component (C) (water-soluble component), and the mixture is heated to the same temperature as the oil-soluble component and slowly added dropwise to the oil-soluble component phase. The mixture is stirred to uniformity while maintaining the temperature.

### (When Physiologically Active Substance is Hydrophilic)

Component (A), a water-soluble physiologically active substance as component (B) and, where necessary, polyol are added to water as component (C) (water-soluble component), and the mixture is heated and mixed to completely and uniformly dissolve them. The heating temperature is generally 60-80° C., preferably 70-80° C. Where necessary, the water-soluble component phase is slowly added dropwise to a non-ionic surfactant and a multilamellar vesicle forming aid, which were heated, dissolved and mixed at the same temperature. Then, the mixture is stirred to uniformity while maintaining the temperature.

### (When Lipophilic and Hydrophilic Physiologically Active Substance is Combinedly Used)

A lipophilic component (B) and, where necessary, a non-ionic surfactant and/or a multilamellar vesicle forming aid are heated and mixed to completely and uniformly dissolve them (oil-soluble component). The heating temperature is generally 60-80° C., preferably 70-80° C. Then, component (A), hydrophilic component (B) and, where necessary, polyol are added to water as component (C) (water-soluble component), and the mixture is heated to the same temperature as the oil-soluble component and slowly added dropwise to the oil-soluble component phase. The mixture is stirred to uniformity while maintaining the temperature.

Examples of the stirring apparatus include paddle mixer, homodisper, homogenizer and the like. The stirring rate is generally 500-5000 rpm, preferably 1500-3000 rpm. The stirring time is generally 5-20 min, preferably 10-15 min. The temperature of the system is gradually cooled to about 40° C. with gentle stirring to give the object multilamellar vesicle preparation.

The multilamellar vesicle preparation produced by the aforementioned method can be adjusted to fine particles having a uniform particle size of the multilamellar vesicles by using, as necessary, an extruder, a high-pressure emulsifier, ultrasonic wave and the like.

The particle size of the multilamellar vesicle is generally 25-10000 nm, preferably 50-3000 nm, more preferably 80-2500 nm. The particle size can be measured by a conventional method and generally using a particle size analyzer.

While it is possible to use the thus-obtained multilamellar vesicle preparation as it is, the preparation may be mixed with known additives and the like as necessary and formulated as a pharmaceutical product, a food or drink, a quasi-drug, a feed and the like by a conventional method.

In addition, an external preparation or cosmetic containing the aforementioned multilamellar vesicle preparation is also another embodiment of the present invention. The external preparation or cosmetic of the present invention can be produced by mixing with known additive and the like as necessary and by a conventional method.

As the additive, water-soluble component, oily component, powder component, surfactant, polymer component, thickener, adhesiveness improver, film-forming agent, pH adjuster, antioxidant, sterilizer, antimicrobial agent, preservative, firmness agent, moisturizer, skin protector, algefacient, flavor, colorant, chelating agent, lubricant, anti-inflammatory agent, antipruritic agent, blood circulation promoter, astringent, tissue repair promoter, adiaphoretic, inorganic or organic powder, ultraviolet absorber, plant extraction component, animal extraction component and the like can be blended as appropriate as long as the effect of the present invention is not inhibited.

Examples of the external preparation include cream, liquid, lotion, emulsion, tincture, ointment, aqueous gel, oily gel, aerosol, powder, shampoo, soap, enamel for coating nails and the like.

Specific examples of the cosmetics include basic cosmetic (e.g., skin lotion, milky lotion, makeup base, serum, night cream, facial mask, makeup remover product (cleansing gel etc.), nail cream etc.), sun care product (e.g., sunscreen, lotion for sunburn skin etc.), hair treatment agent (e.g., hair treatment, out-bath treatment, serum for hair, split end mender etc.), hair styling products (e.g., brushing lotion, curler lotion, pomado, stick pomade, hair spray for styling, hair mist, hair liquid, styling foam, hair gel, water grease etc.), shaving product (e.g., shaving cream, after-shave lotion etc.), makeup cosmetic (e.g., foundation (solid, cream, liquid etc.), BB cream, CC cream, concealer, rouge, lip gloss, eye shadow, eyeliner, blush, mascara, bronzer etc.), perfumes, lip cream, adiaphoretic, oral cosmetic, tooth paste, bath cosmetic (e.g., bathing powder, bath salt etc.) and the like.

The content of the multilamellar vesicle preparation of the present invention to be contained in a cosmetic or external preparation is appropriately determined according to the dosage form, object and the like.

Other features of the invention will become apparent in the course of the following descriptions of exemplary embodiments which are given for illustration of the invention and are not intended to be limiting thereof.

### EXAMPLES

The present invention is concretely explained in the following by referring to Production Examples and Examples, which are not to be construed as limitative.

### Production Example 1

### Synthesis of bis(N^{ε}-lauroyl-L-lysine)sebacoyl amide disodium salt

N^{ε}-lauroyl-L-lysine (8.2 g, 25 mmol) was dissolved in water (70 g) and 25% aqueous sodium hydroxide solution (10 g), and diethylether (80 g) was added. Sebacoyl chloride (3.3 g, 14 mmol) was slowly added to the ether layer. The two-layer solution was stirred for about 1 hr while maintaining at 0° C., and then stirred at room temperature for 23 hr. A 75% sulfuric acid was added dropwise to adjust to pH 2, and the obtained white precipitate was collected by filtration, washed well with water and dried. The obtained compound was dissolved in aqueous sodium hydroxide solution to give an aqueous solution of 10% bis(N^{ε}-lauroyl-L-lysine)sebacoyl amide disodium salt.

### Production Example 2

### Synthesis of acetyl ethylcarboxyl methylthiazolidine carboxylic acid

L-cysteine hydrochloride monohydrate (100 g, 569 mmol) was dissolved in water (200 ml), and 6N aqueous sodium hydroxide solution was added to adjust to pH 5.03. The reaction mixture was heated to 40° C., pyruvic acid ethylester (76 ml, 684 mmol) was gradually added, and the mixture was stirred at 40° C. for 3.5 hr to give 2-methylthiazolidine-2,4-dicarboxylic acid-2-ethyl ester. After completion of the reaction, the mixture was extracted with ethyl acetate, washed with saturated brine, and dried over anhydrous magnesium sulfate. Triethylamine (159 ml, 1141 mmol) was added to the obtained ethyl acetate solution under argon, acetyl chloride (61 ml, 858 mmol) was slowly added dropwise, and the reaction mixture was heated under reflux for 4 hr to give acetyl ethylcarboxyl methylthiazolidine carboxylic acid. After completion of the reaction, water (300 ml) was added, and pH was adjusted to pH 1.0 with HCl. The aqueous layer was separated, and the organic layer was washed with water (300 ml), and then with saturated brine, and dried over anhydrous magnesium sulfate. The obtained ethyl acetate solution was concentrated to about 500 g, and heptane was added to allow for recrystallization. The crystals were washed with heptane/ethyl acetate=2/1, and dried under reduced pressure at 50° C. to give acetyl ethylcarboxyl methylthiazolidine carboxylic acid as crystals (81 g, yield 55%).

¹H-NMR (CDCl₃): δ; 1.27 (3H, t, J=7.12 Hz), 1.94 (3H, s), 2.18 (3H, s), 3.40 (1H, d, J=11.6 Hz), 3.56 (1H, dd, J=5.5, 11.0 Hz), 4.20 (2H, t, J=7.08 Hz), 5.00 (1H, d, J=5.9 Hz), 9.10 (1H, brs).

### <Preparation Method 1>

The compound of component (B), a non-ionic surfactant, and a multilamellar vesicle forming aid in the amounts shown in Table 1 were completely and uniformly dissolved and mixed at 80° C. Then, the compound of Production Example 1 as component (A) (10% aqueous solution) and polyol were added to water as component (C), and the mixture was heated to 80° C. and slowly added dropwise to the oil-soluble component phase. Using homodisper, Tokushukika Corporation (now PRIMIX Corporation), the mixture was stirred at 2500 rpm, 80° C. for 5 min, and the temperature of the system was gradually lowered to about 40° C. to give a multilamellar vesicle preparation.

### <Preparation Method 2>

Separately, a composition was obtained by the same method as in Preparation Method 1 except that the above-mentioned component (A) was not added as described in Table 2.

### <Evaluation Method>

### Presence or Absence of Formation of Multilamellar Vesicle Structure

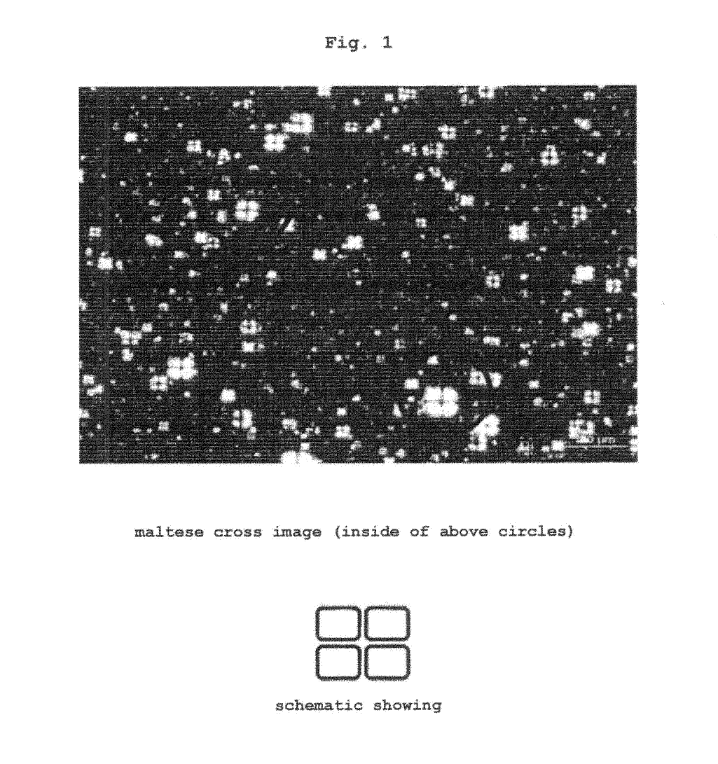

Evaluation was made based on whether or not the presence of a maltese cross image could be confirmed by observation under a polarization microscope (manufactured by Nikon, magnification 400×). As shown in FIG. 1, one with a confirmed maltese cross image was judged to have formed a multilamellar vesicle structure. The evaluation criteria are as described below.

⊙: maltese cross image can be confirmed on the whole.
◯: maltese cross image can be confirmed partially.
x: maltese cross image cannot be confirmed.

### Preservation Stability Test

The prepared compositions were each preserved for 2 weeks in a thermostatic tank at 40° C., 25° C., −5° C., and subjected to observation with a polarization microscope. One with a confirmed maltese cross image was judged to have formed a multilamellar vesicle structure. The evaluation criteria are as described above.

TABLE 1

| component name | | Ex. 1 | Ex. 2 | Ex. 3 | Ex. 4 | Ex. 5 | Ex. 6 |
|---|---|---|---|---|---|---|---|
| (A) compound of Production Example 1 (10% aqueous solution) | | 15 | 10 | 10 | 12 | 15 | 15 |
| (B) physiologically active | tocopheryl acetate | 0.5 | | | | | 0.1 |
| substance | compound of Production Example 2 | | 0.2 | | | | |
| | retinyl palmitate | | | 0.1 | | 0.1 | |
| | ascorbyl tetra-2-hexyldecanoate | | | | 0.1 | | |
| | allantoin | | | 0.1 | | | |
| | menthol | | | | 0.1 | | |
| | guaiazulene | | | | | 0.02 | |
| | oil-soluble licorice extract | | | | | | 0.1 |
| polyol | DPG | | 10 | 10 | 5 | 5 | |
| | BG | | | 5 | 5 | 10 | 10 |
| multilamellar vesicle | cholesteryl isostearate | | 5 | 0.5 | 2 | | 0.2 |
| forming aid | phytosteryl/octyldodecyl lauroyl | | 0.25 | 0.2 | | 0.5 | 0.3 |
| | glutamate | | | | | | |
| non-ionic | Polysorbate 80 | | | 2 | 1 | | |
| surfactant | PEG-40 Hydrogenated Castor Oil PCA | | | | | 2 | |
| | Isostearate | | | | | | |
| | polyoxyethylene hydrogenated castor oil | | | | 1 | | 2 |
| | (60EO) | | | | | | |
| (C) water | | 84.5 | 74.55 | 72.1 | 73.8 | 67.38 | 72.3 |
| total | | 100 | 100 | 100 | 100 | 100 | 100 |
| immediately after adjustment | ability to form multilamellar vesicle | ⊙ | ⊙ | ⊙ | ⊙ | ⊙ | ⊙ |
| preservation stability | 40° C. | ⊙ | ⊙ | ⊙ | ⊙ | ⊙ | ⊙ |
| | 25° C. | ⊙ | ⊙ | ⊙ | ⊙ | ⊙ | ⊙ |
| | 5° C. | ⊙ | ⊙ | ⊙ | ⊙ | ⊙ | ⊙ |

DPG; dipropyleneglycol, BG; butyleneglycol unit in mass %

TABLE 2

| | | Com. | Com. | Com. | Com. | Com. | Com. |
|---|---|---|---|---|---|---|---|
| component name | | Ex. 1 | Ex. 2 | Ex. 3 | Ex. 4 | Ex. 5 | Ex. 6 |
| (A) compound of Production Example 1 (10% aqueous solution) | | | | | | | |
| (B) physiologically active | tocopheryl acetate | 0.5 | | | | | 0.1 |
| substance | compound of Production Example 2 | | 0.2 | | | | |
| | retinyl palmitate | | | 0.1 | | 0.1 | |
| | ascorbyl tetra-2-hexyldecanoate | | | | 0.1 | | |
| | allantoin | | | 0.1 | | | |
| | menthol | | | | 0.1 | | |
| | guaiazulene | | | | | 0.02 | |
| | oil-soluble licorice extract | | | | | | 0.1 |
| polyol | DPG | | 10 | 10 | 5 | 5 | |
| | BG | | | 5 | 5 | 10 | 10 |
| multilamellar vesicle forming | cholesteryl isostearate | | 5 | 0.5 | 2 | | 0.2 |
| aid | phytosteryl/octyldodecyl lauroyl | | 0.25 | 0.2 | | 0.5 | 0.3 |
| | glutamate | | | | | | |
| non-ionic surfactant | Polysorbate 80 | 1 | 1 | 2 | 1 | | 1.5 |
| | PEG-40 Hydrogenated Castor Oil PCA | | 1 | | 1 | 2 | |
| | Isostearate | | | | | | |
| | polyoxyethylene hydrogenated castor | 1 | | 1 | 1 | 2 | 2 |
| | oil (60EO) | | | | | | |
| (C) water | | 97.5 | 82.55 | 81.1 | 84.8 | 80.38 | 85.8 |
| total | | 100 | 100 | 100 | 100 | 100 | 100 |
| immediately after preparation | ability to foim multilamellar vesicle | x | x | x | x | x | x |
| preservation stability | 40° C. | x | x | x | x | x | x |
| | 25° C. | x | x | x | x | x | x |
| | 5° C. | x | x | x | x | x | x |

DPG; dipropyleneglycol, BG; butyleneglycol unit in mass %

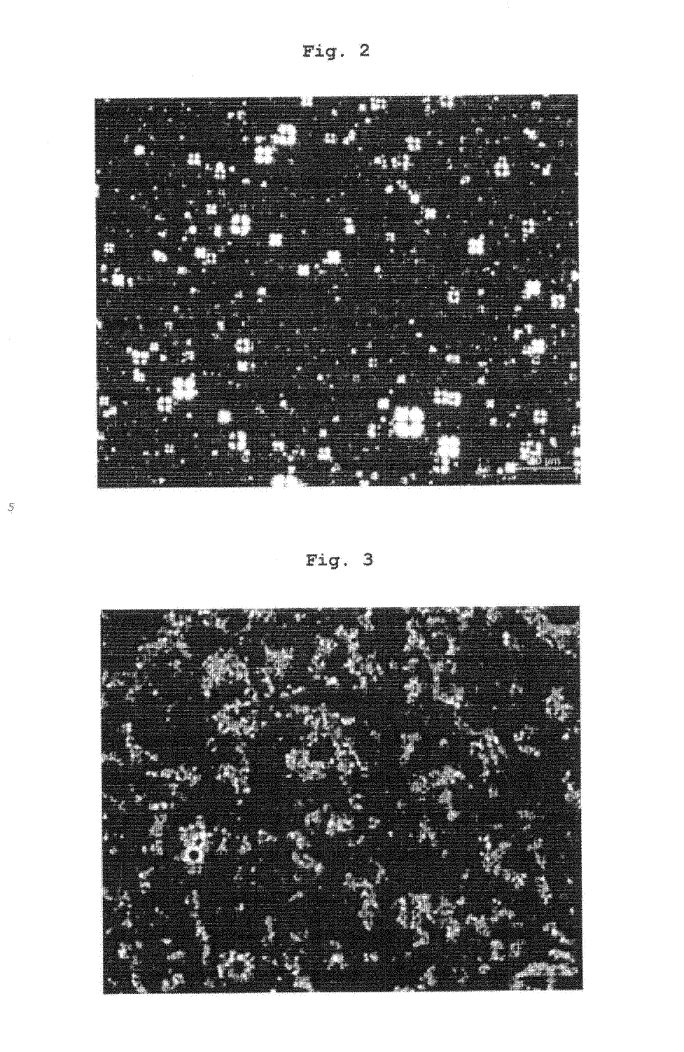

As shown in Table 1, a multilamellar vesicle preparation forming a multilamellar vesicle structure and superior in preservation stability was obtained with components (A)-(C) alone (Example 1). Furthermore, when polyol, a non-ionic surfactant, and a multilamellar vesicle forming aid were added, a multilamellar vesicle preparation forming a multilamellar vesicle structure and superior in preservation stability was obtained as shown in FIG. 2 (Example 2). In contrast, when component (A) was not added, a multilamellar vesicle structure was not formed as shown in Table 2 and FIG. 3 (Comparative Example 2).

### INDUSTRIAL APPLICABILITY

The present invention can provide a multilamellar vesicle preparation which can be produced conveniently, is superior in preservation stability, and can be used for various applications such as external preparation, cosmetics and the like.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

This application is based on a patent application No. 2016-009785 filed in Japan, the contents of which are incorporated in full herein.

1. A multilamellar vesicle preparation, comprising:
(A) a compound represented by formula (1): 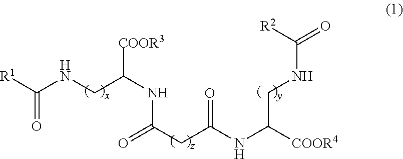
wherein
R¹ and R² are each independently an alkyl group having 5 to 21 carbon atoms or an alkenyl group having 5 to 21 carbon atoms,
R³ and R⁴ are each independently a hydrogen atom, an alkyl group having 1 to 22 carbon atoms, or an alkenyl group having 2 to 22 carbon atoms,
z is an integer of not less than 0, and
x and y are each independently an integer of 2 to 4, or a salt thereof;
(B) a physiologically active substance; and
(C) water.

2. The preparation according to claim 1, wherein, in said formula (1), z is an integer of 0 to 10, or a salt thereof.

3. The preparation according to claim 1, wherein, in said formula (1), z is 7 or 8.

4. The preparation according to claim 1, wherein, in said formula (1), x and y are each 4.

5. The preparation according to claim 1, wherein, in said formula (1), R¹ and R² are each independently a straight chain alkyl group having 5 to 15 carbon atoms.

6. The preparation according to claim 1, wherein, in said formula (1), both R³ and R⁴ are hydrogen atoms.

7. The preparation according to claim 1, wherein (A) said compound of formula (1) or salt thereof is bis(N^{ε}-lauroyl-L-lysine)sebacoyl amide or a salt thereof.

8. The preparation according to claim 1, wherein (B) said physiologically active substance is at least one member selected from the group consisting of a whitening agent, an antioxidant, an anti-inflammatory agent, an algefacient, an animal-derived component, and a plant-derived component.

9. The preparation according to claim 1, wherein (B) said physiologically active substance is at least one member selected from the group consisting of tocopheryl acetate, acetyl ethylcarboxyl methylthiazolidine carboxylic acid, retinyl palmitate, ascorbyl tetra-2-hexyldecanoate, allantoin, menthol, guaiazulene, and an oil-soluble licorice extract.

10. The preparation according to claim 1, which comprises 0.0001 to 2 parts by weight of (B) said physiologically active substance per 1 part by weight of (A) said compound of formula (1) or salt thereof.

11. The preparation according to claim 1, which comprises 30 to 200 parts by weight of (C) said water per 1 part by weight of (A) said compound of formula (1) or salt thereof.

12. An external preparation, comprising a preparation according to claim 1.

13. A cosmetic, comprising a preparation according to claim 1.

14. A method of treating skin, comprising applying a preparation according to claim 1 to skin.

15. The method according to claim 14, wherein said treating is whitening said skin.

16. A method of treating skin, comprising applying a cosmetic according to claim 13 to skin.

17. The method according to claim 16, wherein said treating is whitening said skin.
